(19) United States
(12) Patent Application Publication
(10) Pub. No.: US 2023/0211152 A1
(43) Pub. Date: Jul. 6, 2023

(54) DEVICE AND METHOD FOR ELECTROTHERAPY AND/OR ELECTROPHYSIOLOGY

(71) Applicant: WISE SRL, Milano (IT)

(72) Inventors: Alessandro Antonini, Milano (IT); Marco Vidotto, Seveso (IT); Federica Montrone, Andria (IT); Matteo Saini, Desio (IT); Omar Caspani, Carugo (IT); William David Cordova Jimenez, Cambridge (GB); Sergio Malorni, Oakington (GB); Stuart James Curtis, Sawtry (GB); Brian Flint, Raleigh, NC (US); Matthew Robert Penny, Holly Springs, NC (US)

(21) Appl. No.: 17/835,087

(22) Filed: Jun. 8, 2022

(30) Foreign Application Priority Data
Jan. 5, 2022 (IT) 102022000000155

(51) Int. Cl. A61N 1/05; A61N 1/372

(52) U.S. Cl. CPC A61N 1/0553; A61N 1/37205

(57) ABSTRACT

A kit comprises a device (1) for electrotherapy and/or electrophysiology and a delivery system and method for the device. The device (1) includes a lead (2) and a paddle (5) comprising at least one electrode (8), the paddle (5) being reconfigurable between an operative configuration and a transport configuration of smaller transverse extent. The delivery system comprises a flexible, hollow outer sheath (28) and a hollow delivery sheath (100) for receiving at least the paddle (5) of the device (1) in the transport configuration. The delivery sheath (100) may be inserted into a proximal end of the outer sheath (28) and transported through the outer sheath (28) to deliver the paddle (5) of the device (1) to a distal end of the outer sheath (28), which is located at or directed towards an implantation site in the body (11) of a patient. The delivery sheath (100) protects the paddle (5) during transport and permits enhanced control of the position and orientation of the paddle (5) when it is deployed.

This application claims benefit of Italian Patent Application No. IT102022000000155, filed Jan. 5, 2022, which is hereby incorporated by reference in its entirety.

### FIELD OF INVENTION

The present invention relates to devices for electrotherapy and/or electrophysiology and to systems and methods for delivering such devices into the body of a patient, for example adjacent to the spinal cord. In particular, the devices may be used as part of a stimulator for electrotherapy or as part of a monitor for electrophysiology. Applications include neuromodulation, neuromonitoring, neuroprosthetics and brain-machine interfaces.

### BACKGROUND OF THE INVENTION

Minimally invasive surgical procedures for the delivery of an implantable device into the human body are generally known in the art. For example, it is known to use interventional catheters for accessing the target living anatomy through, for example, a venous port.

It is also known to deploy implantable devices that are able to reduce their radial size when in a transport configuration to fit the lumen of an interventional catheter and eventually to increase their radial size when the implantation site is reached. These known implantable devices are generally controlled by control wires extending all way through a longitudinal lumen of the catheter in such way to allow an operator, typically a surgeon or other clinician, to control deployment of the implantable device from a control station — for example a catheter handle — located outside the patient’s body.

Shape memory materials, for example shape memory alloys like nitinol and/or shape-memory cross-linked polymers, are also generally used for biomedical implants and have the ability to restore their original shape when subject to suitable thermal stress. For example, self-expanding stent grafts and other radially expansible implantable structures are typically made of nitinol, in order to have the property of self-expanding when implanted in a living anatomy due to the temperature increase within the patient’s body. Shape-memory cross-linked polymers typically achieve the shape memory effect owing to a melting transition from a hard to a soft phase, substantially like a glass material. Super-elastic materials, commonly referred to as pseudo-elastic materials, are also known in the art and exhibit the ability to undergo extremely large elastic reversible deformation without for that reason requiring a thermal activation to achieve their original shape.

In the technical field of electrophysiology, it is known to provide implantable electrodes for detecting the electrical activity of a living anatomy. For example, the electrical activity of the brain may be detected by means of needle electrodes inserted in the patient’s scalp. A control unit comprising a recording device is typically associated with the implantable electrodes for recording and filtering the detected signal, for example an electro-corticography signal.

Implantable electrodes for electrotherapy are also known in the art. For example, artificial cardiac pacing is commonly achieved by means of implantation of an active device able to transfer a stimulation pattern to the contractile tissue of the heart in order to control the heart rate.

In the technical field of neuromodulation, implantable electrodes are used to induce a controlled alteration of the function of a nervous tissue by means of applying specific electric and/or magnetic stimulation patterns. Neuromodulation treatments also include applications in medication-resistant epilepsy, chronic head pain conditions and functional therapy ranging from bladder and bowel or respiratory control to improvement of sensory deficits, such as hearing (cochlear implants and auditory brainstem implants) and vision (retinal implants). For example, peripheral nerve stimulation of the occipital nerves aims to relieve chronic migraine pain.

Moreover, the technical field of brain-computer interfaces uses implantable electrodes for providing a two-way direct communication between a brain and a device, mainly for neuroprosthetics, in order to restore damaged movement, sight and/or hearing. The two-way communication requires some implantable electrodes to act as stimulation electrodes for transmitting electrical stimuli and some others as recording electrodes for sensing the electrical activity of the target living anatomy. Therefore, a plurality of such electrodes are usually arranged in an array form.

In addition, in the field of cardiac resynchronization therapy, implantable electrodes are employed. Typically, these electrodes comprise stimulation electrodes to transfer the electrical impulses to the heart as well as recording electrodes to detect information about the electrical state and activity of the heart.

In one example, it is generally known to implant electrodes for spinal cord stimulation (SCS) aiming to relieve chronic pain. These stimulation electrodes are inserted in the epidural space, which is a channel extending along the vertebral foramen of the spinal column and behind the spinal cord in the sagittal plane of the patient’s body.

As the need is felt to introduce an implantable electrode within a target living anatomy with the purpose of electrical interaction with the tissue of the target living anatomy, it is desirable that the electrode should be implanted through a narrow access that necessarily limits the maximum size of the electrode. The access is normally but not necessarily via a percutaneous incision.

It is known in the art to provide deployable electrodes for living tissue stimulation that are able to increase their transverse size when unconstrained by the delivery catheter or a part thereof. Such deployable electrodes usually comprise springs for biasing the body of the deployable structure to increase their transverse size upon deployment. For example, U.S. Pat. Application Publication No. 2008/140152 shows a transversally foldable paddle electrode having a body of the implant structure made of a thin film of flexible circuitry. This solution allows the paddle to deploy after having reached the target tissue. However, this solution results in poor manoeuvrability when the paddle electrode is unconstrained by the delivery catheter or a part thereof, due its high flexibility.

PCT Publication No. WO 2011/121017 and European Patent Application No. EP 2553135, in the name of the present applicant, disclose a technique for production of electrically functionalized stretchable articles, by means of the burial of nano-metric neutral particles beneath the free surface of an elastic flexible substrate and within the core of the substrate. Moreover, PCT Publication No. WO 2017/203441, also in the name of the present applicant, describes a technique to connect an intrinsically stretchable functionalized conductive polymer to a rigid electrical conductor.

U.S. Pat. No. 6,714,822 shows a rigid lead tip having a plurality of flexible paddle electrodes transversally extending therefrom. During transport through a delivery catheter, said flexible paddle electrodes are folded around the rigid tip. This solution allows the paddle electrodes to deploy through a pivoting movement of the entire lead about its longitudinal axis. However, the proposed deployment strategy generates torque stress along the lead during deployment. Moreover, when in transport configuration within a delivery catheter, the paddle electrodes comprising metal conductive lines cannot bend beyond a certain curvature radius without compromising the structural integrity of the metal, therefore requiring the provision of void volumes inside the sheath. Thus, the ratio of the delivery sheath diameter to the paddle width is high.

In some applications, for example when the paddle electrodes are deployed alongside the spinal cord, the orientation of the paddle about the longitudinal axis of the lead may be important so that the paddle electrodes will apply stimulation or to monitor electrical activity in the correct location. In the system disclosed in U.S. Pat. No. 6,714,822, a pivoting movement of the entire lead about its longitudinal axis is used to deploy the electrodes from their transport configuration, therefore such a pivoting movement cannot also be used to set the desired orientation of the paddle about the axis.

PCT Publication No. WO 2020/201252, in the name of the present applicant, discloses a device for electrotherapy and/or electrophysiology comprising at least one lead having an elongated lead body extending along a longitudinal direction and a paddle comprising at least one paddle electrode configured to come into electrical contact with a living anatomy inside a patient’s body. Means for electrically connecting the electrodes to the lead are disclosed and, during delivery of the paddle to the implantation site, the paddle electrodes may be configured in an “omega” shape to reduce their transverse size. The paddle may be delivered by sliding the lead through a sheath that has previously been introduced into the anatomy of the patient. The sheath must be sufficiently stiff to overcome high frictional and compressive forces from the patient’s tissues as the distal end of the sheath is pushed towards the implantation site, which limits the manoeuvrability of the sheath for precise positioning of the paddle electrodes. Also, there is a risk of damage to the electrodes as the paddle slides past the robust material of the sheath.

### SUMMARY OF THE INVENTION

The invention provides a kit comprising a device for electrotherapy and/or electrophysiology and a delivery system for the device;
wherein the device comprises:
   a lead; and
   a paddle comprising a paddle electrode having an exposed surface configured to come into contact with living anatomy inside a patient’s body;
   the paddle being reconfigurable between a transport configuration and an operative configuration, a transverse extent of the paddle being smaller in the transport configuration than in the operative configuration; and
wherein the delivery system comprises:
   a flexible, hollow outer sheath comprising a proximal end and a distal end; and
   a hollow delivery sheath for receiving at least the paddle of the device in the transport configuration;
   wherein the delivery sheath is configured to be inserted into the proximal end of the outer sheath and transported through the outer sheath to deliver the paddle of the device to the distal end of the outer sheath.

Accordingly, delivery of the paddle to the implantation site involves sliding the delivery sheath through the outer sheath. The paddle does not need to slide relative to the delivery sheath, except during insertion into the delivery sheath and ejection from the delivery sheath. Therefore, the design and choice of materials of each component can be optimized for the limited functions that it needs to perform. The delivery sheath can be soft and flexible to avoid damage to the paddle and the paddle electrodes and to accommodate the changing shape of the paddle as it transitions between the transport and operational configurations. The soft material also makes the delivery sheath easier to manoeuvre when advancing from the distal end of the outer sheath towards the implantation site, with minimal damage to the surrounding anatomy. In addition, it can have a small outer diameter in comparison to the sheath of the prior art, making it easier to advance through tighter spaces and overcome obstacles in the anatomy. The outer sheath can be made relatively stiff and strong to withstand the frictional and compressive forces it is subjected to and resist buckling while being inserted into the tissue of the patient. In some cases, the outer sheath can be made of multiple materials with different characteristics, e.g. the tip can be soft to avoid tissue damage and part of the length can be stiffer to avoid buckling.

The material of the delivery sheath can also be chosen to slide through the outer sheath and rotate in the outer sheath with minimal friction, while the paddle does not need to be designed for such sliding contact. The low friction between the delivery sheath and the outer sheath will enable the operator to have a very clear feedback of the forces experienced and applied inside the patient’s body while advancing the delivery sheath and paddle inside the body, for example in the epidural space. This makes the procedure safer compared with the prior art, in which the forces experienced when advancing the sheath towards the implantation site were dominated by the compression forces from adjacent tissues.

In some embodiments, the delivery sheath comprises a proximal end and a distal end; the distal end comprising a distal opening, through which the paddle is capable of being received and ejected. This contrasts with the prior art, in which, during implantation, the paddle is inserted into the proximal end of the sheath and ejected from the distal end of the sheath, the reverse being true during explantation. Therefore, in the prior art, the paddle needs to be designed to be capable of folding or unfolding when either end of the paddle is compressed by the rim of the sheath. In this preferred embodiment of the present invention, it is always the proximal end of the paddle that is inserted into or ejected from the distal end of the delivery sheath, therefore the design of the paddle can be simplified.

The delivery sheath may further comprise a proximal opening in the proximal end of the delivery sheath, through which the lead of the device is capable of passing. This is a convenient arrangement enabling relative longitudinal movement between the delivery sheath and the lead, which results in the paddle being inserted into or ejected from the distal end of the delivery sheath. In one application, the operator might be able to receive a packaged device in which the lead extends through the proximal opening of the delivery sheath and the paddle is not inserted into the distal end of the delivery sheath. Thus, the paddle would not be folded into its transport configuration before use, which could compromise the resilience or the electrical connection of the paddle during storage of the device over a long time. It would also allow the operator to inspect the paddle visually before use. Then the operator would take this delivery sheath and hold it while pulling on the proximal end of the lead, which would enable the paddle to fold and be inserted into the distal end of the delivery sheath without the operator ever touching the paddle. This has the benefit of avoiding the unpredictable and potentially damaging forces that an operator might impose on the paddle when folding it manually, while sterility is maintained because the operator does not touch the paddle itself.

In some embodiments, the paddle is configured to adopt its operative configuration automatically when ejected from the delivery sheath. This may be achieved, for example, by making the paddle from a resilient material, by embedding springs or other resilient structures in the construction of the paddle or by the use of shape memory materials. The alternative is to provide arrangements for changing the configuration of the paddle under the manual control of the operator, for example through mechanical, electrical or hydraulic means. While this permits greater control over the timing of the transition, it adds to the complexity of the device. The ability to use the delivery sheath to position the paddle correctly before it is deployed, in accordance with the present invention, reduces the advantages of such control arrangements.

In some embodiments of the invention, the device further comprises a stylet for controlling movement of the lead from outside the body. The stylet enables the operator to push the lead and paddle forwards and to steer a distal end of the lead towards the implantation site.

The device may further comprise at least one fluoro-marker, by which the orientation of the device may be determined using fluoroscopy. It is an advantage of the use of a delivery sheath in accordance with the present invention that, while the paddle remains at least partially within the delivery sheath, the delivery sheath can be rotated within the outer sheath to adjust the orientation of the paddle. If the at least one fluoro-marker is provided on the paddle, then the paddle may first be partially ejected from the delivery sheath in order that it starts to unfold and the orientation of the device becomes clear. In some embodiments, at least one fluoro-marker may be provided on the lead so that the orientation of the device is already apparent while the paddle remains in the transport configuration.

In some embodiments of the invention, the delivery system further comprises: a substantially rigid, hollow needle for creating a path into the body of the patient; a guidewire capable of passing through the needle; and a hollow dilator capable of engagement around the guidewire and movement along the guidewire; wherein the hollow outer sheath is capable of engagement around the dilator to move with it along the guidewire. These components provide a convenient means for introducing the outer sheath into the body such that its distal end is at or directed towards the desired implantation site.

The invention further provides a method of delivering a device for electrotherapy and/or electrophysiology into the body of a patient;
wherein the device comprises:
   a lead; and
   a paddle comprising a paddle electrode having an exposed surface configured to come into contact with a living anatomy inside a patient’s body;
   the paddle being reconfigurable between a transport configuration and an operative configuration, a transverse extent of the paddle being smaller in the transport configuration that in the operative configuration; and
wherein the method comprises:
   inserting a flexible, hollow outer sheath into the body of the patient such that a distal end of the outer sheath is at or directed towards an implantation site inside the patient’s body;
   inserting at least the paddle of the device into a hollow delivery sheath with the paddle in the transport configuration;
   inserting the delivery sheath into a proximal end of the outer sheath and transporting the delivery sheath through the outer sheath to deliver the paddle of the device to the distal end of the outer sheath; and
   ejecting the paddle from the delivery sheath.

The use of this method of delivering a device provides the advantages previously discussed in relation to the kit, including ease of use and reduced risk of harm to the patient, arising from the design of the outer sheath and the delivery sheath to perform different functions; high manoeuvrability of the delivery sheath; and clear feedback to the operator of the forces experienced during implantation of the device.

In some embodiments, the delivery sheath comprises a proximal end and a distal end; and the insertion of the paddle into the delivery sheath and the ejection of the paddle from the delivery sheath are carried out through a distal opening in the distal end of the delivery sheath. The method may further comprise the step of passing the lead of the device through a proximal opening in the proximal end of the delivery sheath. In some embodiments, the paddle adopts its operative configuration automatically when ejected from the delivery sheath.

The method may also comprise the preliminary steps of:
using a hollow needle to create a path into the body of the patient;
inserting a guidewire through the needle and directing the guidewire such that a distal end of the guidewire is at or directed towards the implantation site; and
withdrawing the needle;
wherein the step of inserting the outer sheath comprises:
engaging the outer sheath around a hollow dilator;
engaging the dilator around the guidewire;
moving the dilator and the outer sheath along the guidewire until a distal end of the dilator is at or directed towards the implantation site; and
withdrawing the dilator through the outer sheath.

In some methods according to the invention, the step of ejecting the paddle from the delivery sheath comprises determining the position or orientation of the paddle by using fluoroscopy to observe at least one fluoro-marker on the device.

Additionally, in some methods according to the invention, the step of ejecting the paddle from the delivery sheath comprises, while the paddle remains at least partially within the delivery sheath, rotating the delivery sheath to adjust the orientation of the paddle. This makes it easier to control the orientation of the paddle because the delivery sheath can slide freely in the outer sheath in the circumferential as well as the longitudinal direction, compared with the prior art in which adjusting the orientation of the paddle would require rotating it against the walls of the outer sheath or against the tissue of the patient.

In summary, the invention provides a device able to fit into a small diameter for the delivery into the body of the patient, while at the same time being able to provide fine-tuned localization of at least one electrode within a living anatomy.

The device used in the present invention is an implantable device. The device may be designed to be implanted long term, perhaps permanently, within the living anatomy of a patient body or may be designed to be implanted temporarily, for example to support surgery being carried out on the patient.

The device used in the present invention may be suitable for electrotherapy applications such as: pain reduction, implanted muscle stimulation, treatment of neuromuscular dysfunctions, tissue repair, treatment of urine and fecal incontinence, treatment of masculine erectile dysfunctions, treatment of edema, lymphatic drainage, peripheral nerve stimulation. The implantable device according to the present invention is particularly, although not exclusively, suitable for spinal cord stimulation.

The device used in the present invention may alternatively be suitable for electrophysiology applications such as: peripheral nerve recording, cardiac wall recording, electro-corticography, electroencephalography, electromyography.

The same device may be used for both electrotherapy applications and electrophysiology applications.

In this specification, the term “distal” describes an end of the device that is relatively more distant from an operator of the device when the device is use, or a direction away from the operator. The term “proximal” describes an end of the device that is relatively closer to the operator when the device is use, or a direction towards the operator.

### BRIEF DESCRIPTION OF THE DRAWINGS

Further characteristics and advantages of the invention will appear from the description below of preferred embodiments, which are given as examples and are not meant to be limiting.

FIGS. 1 and 2 are perspective views of a device according to the prior art, showing the first and second major surfaces of the paddle, respectively.

FIG. 3 is an elevation view of a lead of the device of FIG. 1.

FIG. 4 is an elevation view showing a proximal portion of the lead of FIG. 3 with a control device.

FIGS. 5 and 6 are perspective views of one embodiment of a device according to the present invention, when in a transport configuration and when in an operative configuration, respectively.

FIGS. 7 to 13 illustrate a sequence of steps in a method of implanting a device in a patient, according to some embodiments of the invention.

FIG. 14 is a partial, longitudinal section through a dilator and outer sheath.

FIG. 15 illustrates a paddle being drawn into a delivery sheath in accordance with an embodiment of the invention.

FIG. 16 is a partial, longitudinal section through a device for use in the invention, which comprises a lead stylet.

### DETAILED DESCRIPTION

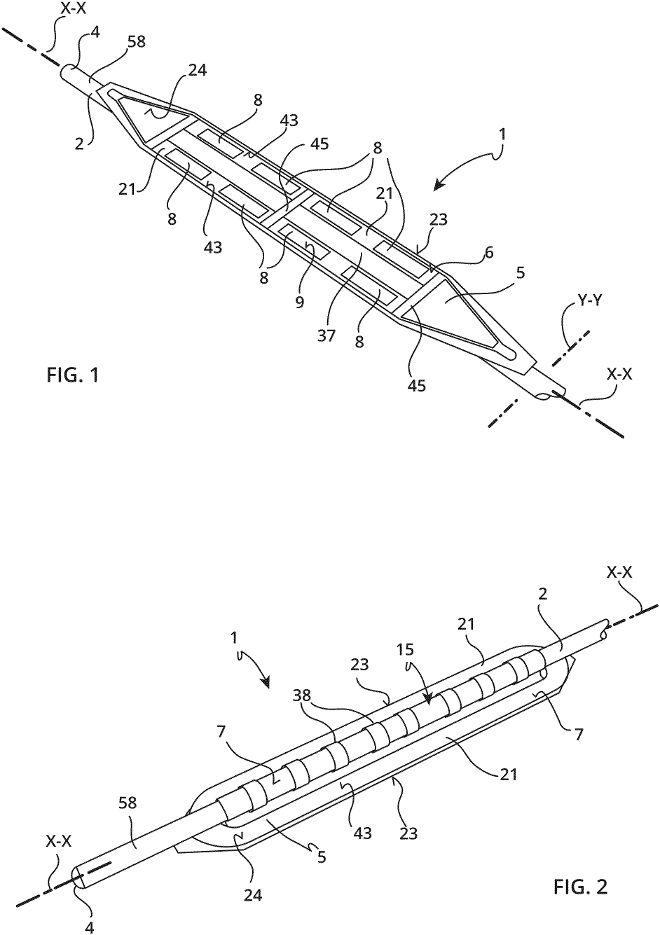

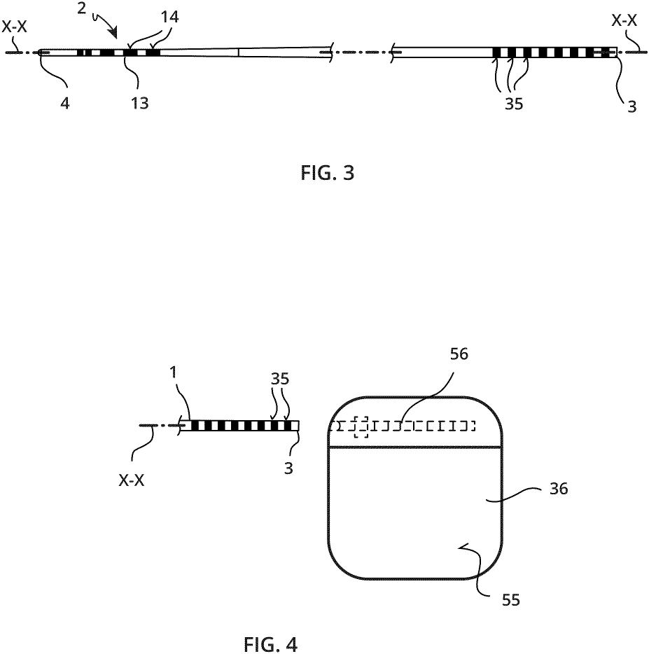

FIGS. 1 to 4 are taken from PCT Publication No. WO 2020/201252, which is hereby incorporated by reference in its entirety, and reference is made to that document for a full description of them.

They show the distal portion of a device 1 suitable for application in electrotherapy or electrophysiology, which comprises a lead 2 and a paddle 5. It would be possible for one or more further paddles (not illustrated) to be arranged along the lead 2. The lead 2 comprises an elongated lead body, which extends along a longitudinal direction X-X and comprises a proximal end 3 and a distal end 4. A transverse direction Y-Y is defined to be orthogonal to said longitudinal direction X-X.

In the illustrated device, at least the distal end 4 of the lead 2 comprises a substantially cylindrical body, in some embodiments having a rounded cross-section around said longitudinal direction X-X. The distal end 4 of the lead 2 may be tapered to reduce the transversal or radial dimension or extent of the device 1.

The paddle 5 has a paddle body comprising two opposite major surfaces 6, 7 defining a paddle thickness therebetween. In some embodiments, the two opposite major surfaces 6, 7 of the paddle 5 face away from one another. Each of the two opposite major surfaces 6, 7 of the paddle 5 comprises a length extending along said longitudinal direction X-X and a width extending along the transverse direction Y-Y. In some embodiments, the paddle thickness is a fraction of each the major surface longitudinal size and/or width. For example, the paddle 5 may be in form of a film or the like.

Said paddle 5 comprises at least one paddle electrode 8 having an exposed surface 9 designed to come into electrical contact with living anatomy 10 inside a patient’s body 11. For example, said living anatomy may comprise a living tissue and/or organ and/or a body fluid. Said exposed surface 9 of the at least one electrode 8 acts as an electrical terminal of the device 1. Thanks to the provision of said at least one electrode 8, said device 1 may provide electrical stimulation to said living anatomy 10 and/or may record electrical activity of a living anatomy 10.

In the illustrated device, said at least one paddle electrode 8 comprises an exposed surface 9 that functionally emerges from said first major surface 6 of the paddle 5. In other words, said exposed surface 9 of said at least one electrode 8 and the first major surface 6 of the paddle body 5 together form the outer surface of the paddle 5. The terminology “exposed surface that functionally emerges from said first major surface” means that the paddle 5 exhibits said exposed surface 9 of the paddle electrode 8. The exposed surface 9 does not necessarily protrude from the first major surface 6, although in some embodiments of the invention the exposed surface 9 does protrude from said first major surface 6. The terminology “exposed surface that functionally emerges from said first major surface” also includes examples wherein said at least one electrode 8 covers the entire first major surface 6 of the paddle 5 forming an exposed surface 9, as well as the case wherein said at least one electrode 8 covers the entire first major surface 6 of a paddle half-body, for example a paddle wing 21. In some embodiments of the invention, the paddle electrodes 8 may comprise exposed surfaces 9 that functionally emerge from both said first and second major surfaces 6,7 of the paddle 5.

The proximal end 3 of the lead 2 comprises one or more electrical contacts 35, which are electrically connected to the one or more paddle electrodes 8 by electrical conductors (not illustrated) that extend along the lead 2. The proximal end 3 of the lead 2 may be received in a port 56 in the case 55 of a control device 36 for providing electrical communication between the control device 36, the electrical contacts 35 of the lead 2 and, in turn, the paddle electrode 8.

In some embodiments of the invention, said control device 36 comprises a pulse generator 36. In this way, a stimulator assembly for electrotherapy is provided. The pulse generator may be implantable.

In some embodiments of the invention, said control device 36 comprises a memory for storing information about the electrical activity of the anatomy 10 of the patient. In this way, a recorder assembly for electrophysiology is provided. The memory may be implantable.

In some embodiments of the invention, said control device 36 comprises a communication unit for transmitting information about the electrical activity of the anatomy 10 of the patient to a device external to the patient and/or for receiving information or instructions from a device external to the patient. The communication unit may be implantable.

The lead 2 comprises a connection portion 13 near the distal end 4 thereof. The connection portion 13 of the lead 2 forms an electrical and mechanical connection with a counter-connection portion 15 of the paddle 5 to secure the paddle 5 to the lead 2 and to provide electrical communication between the lead 2 and the paddle electrodes 8. In the illustrated device, fixing means 37, 38 are provided mechanically connecting said connection portion 13 of the lead 2 and said counter-connection portion 15 of the paddle 5. Said fixing means may include glue 37 connecting said connection portion 13 of the lead 2 and said counter-connection portion 15 of the paddle 5. In some embodiments, said glue 37 is electrically insulating. Said fixing means may further include a plurality of clips 38 mechanically connecting said connection portion 13 of the lead 2 and said counter-connection portion 15 of the paddle 5. In some embodiments, said plurality of clips 38 apply compressive force to create electrical contact between mutually facing electrically conductive surfaces of the lead 2 and the paddle 5.

In some embodiments, the connection portion 13 of the lead 2 radially delimits a longitudinal cavity for hosting a guiding stylet. In this way, the elongated body of at least the portion near the distal end 4 of the lead 2 is longitudinally hollow. In some embodiments, the distal end 4 of the lead 2 longitudinally closes said longitudinal cavity. The guiding stylet provided in said longitudinal cavity may be used to advance the device 1 into the patient’s body 11 and to steer and manoeuvre the device 1 inside the patient’s body 11. In some embodiments, the lead 2 comprises a distal free end portion 58 that is distinct from said paddle 5, on which the stylet may act. In this way, the manoeuvrability of the device 1 is enhanced.

In the illustrated device, said paddle 5 comprises at least one paddle transversal edge 23 delimiting the width of said major surfaces 6, 7. Between the free edge 23 and the counter-connection portion 15 of the paddle 5 is a paddle wing 21, which comprises at least a portion of said at least one paddle electrode 8. According to some embodiments of the invention, said paddle 5 comprises at least two opposite paddle wings 21, each having a free edge 23.

In some embodiments, said at least one paddle wing 21 is able to change its orientation with respect to said lead 2, in order to bring said exposed surface 9 of said at least one paddle electrode 8 to various distances from the lead 2. Furthermore, said at least one paddle wing 21 is able to change its orientation with respect to said lead 2, which enables the exposed surface 9 of the at least paddle electrode 8 to be aligned to rest against a target part of the anatomy 11. In some embodiments, said at least one paddle wing 21 is flexible at least in the transverse direction, so that it is able to conform to the shape of a curved part of the anatomy 11.

Said paddle 5 is able to assume at least one transport configuration and at least one operative configuration, wherein the transverse or radial extent of the paddle 5 when in said at least one transport configuration is less than the transverse or radial extent of the same paddle 5 when in said at least one operative configuration.

The terminology “transverse extent” refers to the maximum dimension of the paddle in the transverse direction Y-Y, while the terminology “radial extension” refers to the maximum dimension of the paddle measured away from the axis X-X. In some embodiments, the transverse extent of the paddle 5 is varied by folding or unfolding the paddle wings 21 along folds that may be generally parallel to axis X-X. When the wings 21 of the paddle 5 are exactly two in number and are opposite to one another with respect to said counter-connection portion 15 of the paddle 5, the cross-section of the device 1 may assume an “omega″-like shape, in other words a Ω-like shape, where the wings 21 are the flat segment of the omega and the counter-connection portion 15 of the paddle 5 is the arched bridge of the omega.

The paddle 5 or at least the one or more paddle wings 21 may be made of a resilient material that naturally tends to unfold towards the operative configuration when the paddle 5 is not constrained in the transport configuration. Additionally or alternatively, said device 1 may comprise at least one biasing structure 24 biasing said paddle 5 towards said operative configuration. Said biasing action is, in some embodiments, an elastic biasing action. Said biasing structure 24 may comprise at least one elongated element 43 forming a closed path, said elongated element 43 being arranged near the transversal edges of the paddle 5. In some embodiments, said elongated element 43 extends along the free edge 23 of the paddle wing 21. Said biasing structure 24 may comprise one or more stiffening elements 45 connected to said at least one elongated element 43. In some embodiments, said one or more stiffening elements 45 are beams extending transversally. Said at least one biasing structure 24 may comprise at least one wire spring, leaf spring or coil spring. In some embodiments of the invention, said at least one biasing structure 24 may comprise at least one shape memory element made of a shape memory material.

By using such a device 1, it is possible to provide localized and directional stimulation to living anatomy 10 and at the same time it is possible to deliver the device 1 to an implantation site inside a patient’s body 11 by means of minimally invasive surgery.

By using such a device 1, it is possible to provide localized and directional recording of the electrical activity of a living anatomy 10 and at the same time it is possible to deliver the device 1 to an implantation site inside a patient’s body 11 by means of minimally invasive surgery.

Such a device 1 may be used in combination with a delivery system for implanting the device into the body 11 of a patient and/or for explanting the device 1 from the body 11 of the patient. In accordance with the present invention, the delivery system comprises an outer sheath 28 and a delivery sheath 100. Advantageously, the delivery sheath 100 houses said device 1 when in transport configuration, during delivery of the paddle 5 of the device 1 to a desired implantation site 1 in the body 11.

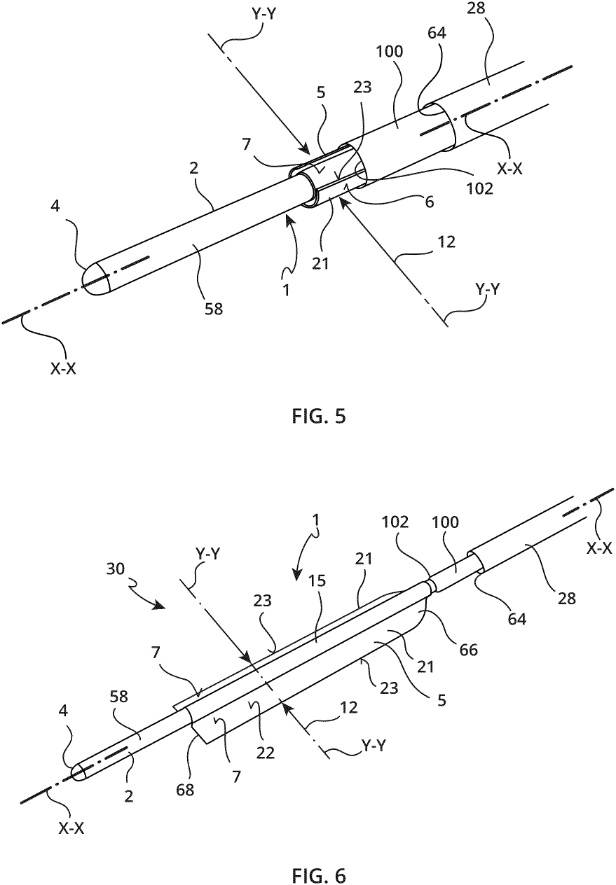

FIG. 5 illustrates, in simplified form, a device similar to that in FIGS. 1 to 4, comprising a paddle 5 electrically and mechanically coupled near the distal end 4 of a lead 2. A distal free end portion 58 of the lead 2 extends beyond the paddle 5, to enhance the manoeuvrability of the device 1 by a stylet that extends through the lead 2. The lead 2 is surrounded by a delivery sheath 100, which is in turn surrounded by an outer sheath 28. The lead 2 is capable of sliding parallel to the longitudinal axis X-X through the hollow delivery sheath 100 and the delivery sheath 100 is capable of sliding parallel to the longitudinal axis X-X through the hollow outer sheath 28. It will be understood that all such movement is relative: for example, the lead 2 may slide within the stationary delivery sheath 100; the delivery sheath 100 may slide along the outside of the stationary lead 2; or both the lead 2 and the delivery sheath 100 may move at the same time.

The outer sheath 28 is a tubular structure, open at both the proximal and distal ends, which can bend sufficiently to change direction as it is inserted in the body 11 of the patient, while being stiff enough not to collapse under the compressive forces it experiences. The proximal end may be provided with a grip for the operator to control the outer sheath 28, in particular to insert it into the body 11 and subsequently to withdraw it. The outer diameter of the outer sheath 28 may taper towards the distal end.

The delivery sheath 100 is also a tubular structure, which is capable of sliding within the hollow outer sheath 28. At the distal end it comprises a distal opening 102, which is able to receive the paddle 5 in the transport configuration. At the proximal end it comprises a proximal opening 104, through which the lead 2 can pass. The proximal opening 104 may be smaller than the distal opening 102. It may comprise a hub (not illustrated) to facilitate the passage of the lead 2 therethrough. The proximal end of the delivery sheath 100 could be provided with additional rigidity, for example with braiding or a coil, to transmit better the torque from the lead 2.

In FIG. 5, the distal end of the delivery sheath 100 protrudes beyond a distal end 64 of the outer sheath 28. The longitudinal position of the lead 2 is such that the paddle 5 is partially surrounded by the delivery sheath 100, which constrains the paddle 5 to remain in its transport configuration. Accordingly, the paddle wings 21 are transversally folded about the lead 2 and the paddle occupies a small transverse extent 12.

In FIG. 6, the lead 2 has been advanced in the distal direction and/or the delivery sheath 100 has been withdrawn in the proximal direction such that the full length of the paddle 5 now extends beyond the distal opening 102 of the delivery sheath 100. This frees the paddle 5 to adopt its operative configuration, with the paddle wings 21 unfolded to their maximum transverse extent 12.

In some embodiments, the paddle 5 is configured to adopt its operative configuration automatically when ejected from the delivery sheath 100, for example by at least the paddle wings 21 being formed of a resilient material or comprising at least one biasing structure 24, as previously described. Conversely, when the paddle 5 is retracted into the delivery sheath 100, contact between the proximal ends 66 of the paddle wings 21 and the rim of the distal opening 102 of the delivery sheath 100 overcomes the bias of the paddle 5 towards the operative configuration and forces the paddle wings 21 to fold inwards into the transport configuration so that they can fit inside the delivery sheath 100. The proximal ends 66 of the paddle wings 21 may be tapered in the distal direction or otherwise shaped to mediate the contact between the paddle wings 21 and the distal end of the delivery sheath 100 and provide a smooth transition in either direction between the transport configuration and the operative configuration. The bias of the paddle 5 towards the operative configuration, combined with a suitable shape of the proximal ends 66 of the paddle wings, may serve to assist the final stage of ejecting the paddle 5 from the delivery sheath 100. Conversely, it may resist the initial stage of withdrawing the paddle 5 into the delivery sheath 100. As will be described below, the paddle 5 is, in some embodiments, ejected from and inserted into the delivery sheath 100 only through the distal opening 102 of the delivery sheath 100, therefore only the shape of the proximal ends 66 of the paddle wings 21 need to be designed with these factors in mind. The distal ends 68 of the paddle wings may be designed solely taking account of other considerations, such as the preferred layout of the paddle electrodes 8 and the minimization of injury to the anatomy 10 of the patient during movement of the paddle 5.

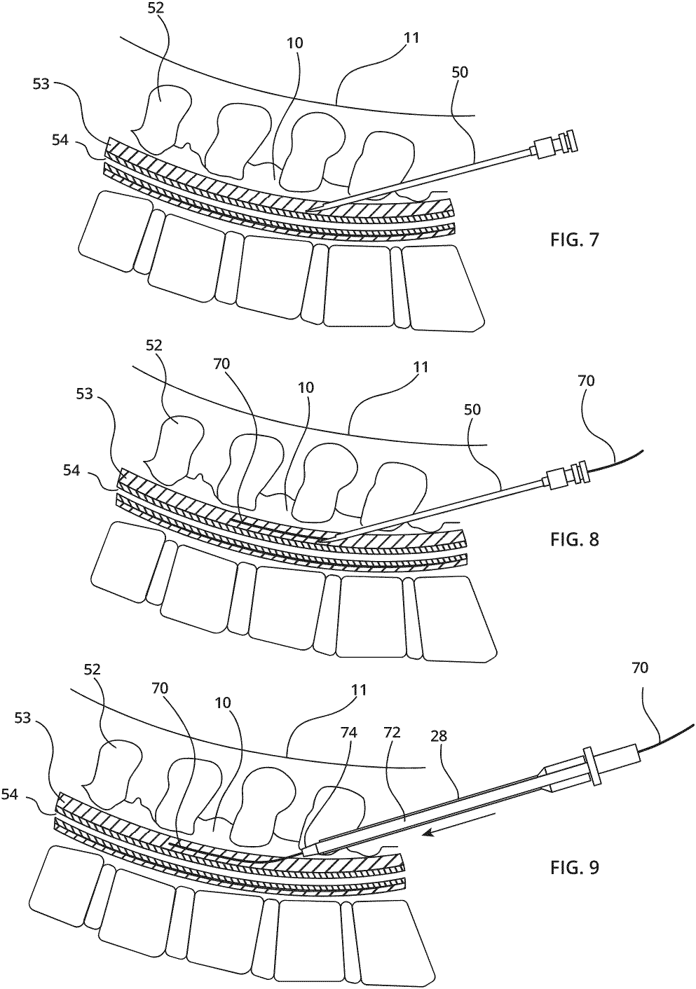

The use of a delivery system to implant the device 1 in the body 11 of the patient will now be described with reference to FIGS. 7 to 13, using the example of implanting a device for spinal cord stimulation (SCS). In SCS, the paddle 5 is implanted in the epidural space 53 adjacent to the spinal cord 54, orientated so that the paddle electrodes 8 are in electrical communication with the spinal cord. In summary, the main procedural steps consist of or comprise:
Insertion of a guidewire into the patient,
Insertion of the outer sheath into the patient,
Outer sheath advancement,
Paddle loading into the delivery sheath,
Delivery sheath advancement,
Controlling orientation of the paddle,
Deploying the paddle at the target site,
Managing position of the paddle,
Removal of the delivery sheath,
Removal of the outer sheath.

The purpose of the guidewire 70 into the patient is to be an element of small diameter, which can be inserted first to define a pathway, which then guides the entry and advancement of a larger diameter dilator 74 and outer sheath 28 to the target location of the outer sheath 28 within the body. The guidewire 70 can be inserted into the patient via an aperture in the tissue or through a device, such as but not limited to, a needle. FIG. 7 shows the insertion of a hollow needle 50, such as a Tuohy needle, through an opening between vertebrae of the spine 52, in some embodiments at a shallow angle (less than 45°). A blade (not shown) may be used to form an initial incision in the skin of the patient, through which the needle 50 is inserted. The needle 50 is inserted until the aperture at the tip of the needle 50 is located in the epidural space, facing towards the desired implantation site for the paddle 5.

FIG. 8 shows the insertion of the guidewire 70 through the needle 50. The guidewire 70 is pushed so that its distal end extends beyond the tip of the needle 50 and travels along the epidural space 53 towards the implantation site for the paddle 5. The guidewire 70 is flexible enough to change direction as it moves from the needle 50 into the epidural space 53. The tip of the guidewire 70 may be placed close to the implantation site. Alternatively, the tip of the guidewire 70 may be placed further from the implantation site: for example, just inside the epidural space so that the outer sheath 28 will be guided by the guidewire 70 only during its initial insertion into the body 11 and will then travel independently along the epidural space 53 towards the implantation site.

It may be preferable for the distal tip of the guidewire 70 to be angled or curved upon entry into the patient’s body 11 to effectively reduce the contact angle between the tip and the tissue compared to a straight tip. This may reduce the force being directly applied to the tissue, thereby reducing the propensity to cause localised damage, and would also reduce the insertion forces experienced by the user. Alternatively, the guidewire 70 may be constructed with a coil to provide flexibility, combined with a central lumen which is open at the proximal end of the guidewire. A guidewire stylet (not illustrated) can be inserted through the lumen to modify the distal shape and overall stiffness of the guidewire. The stylet can have either a straight, angled or curved stylet tip and is inserted at the proximal end of the guidewire until the stylet tip reaches the guidewire tip. In this construction, the guidewire tip would take the shape of the stylet tip. The stylet can then be rotated relative to the guidewire such that the orientation of the curved guidewire tip can change relative to the guidewire body to aid with steering. The curved stylet may optionally be exchanged for a straight stylet when it is desired to push the guidewire 70 along a straight path.

When the guidewire 70 is in the desired position, the needle 50 may be withdrawn. The outer sheath 28 is then introduced over the guidewire 70 to follow the guidewire 70 into the body 11 of the patient. While being introduced into the body 11, the outer sheath 28 is supported by a dilator 74, as shown in FIG. 14.

The dilator 74 consists of or comprises a long, flexible member that slidably fits within the outer sheath 28. A proximal portion of the dilator is designed such that it can be handled by the operator. A distal portion 76 of the dilator 74 is, in some embodiments, shaped such that its cross-sectional area tapers in the distal direction. The outer sheath may similarly be tapered towards its distal end 64. There is a central lumen 78 in the dilator 74 that runs from the proximal end to the distal end. The distal portion 76 of the dilator 74 is, in some embodiments, soft enough to bend —at least through the change of angle where the guidewire 70 enters the epidural space -yet resilient enough to push through an aperture in tissue and dilate it. The outer sheath 28 fits around the dilator 74. The outer sheath 28 and dilator 74 can be pre-assembled or assembled by the operator.

The guidewire 70 is inserted into the dilator’s central lumen 78 and the outer sheath 28 and dilator 74 slide along the pathway defined by the guidewire 70, as shown in FIG. 9. Beyond the point along the pathway where the tip of the dilator 74 first reaches the patient’s tissues, the distal portion 76 of the dilator 74 starts penetrating and progressively dilating the surrounding tissue to form a passage that can accommodate subsequent insertion of the paddle 5.

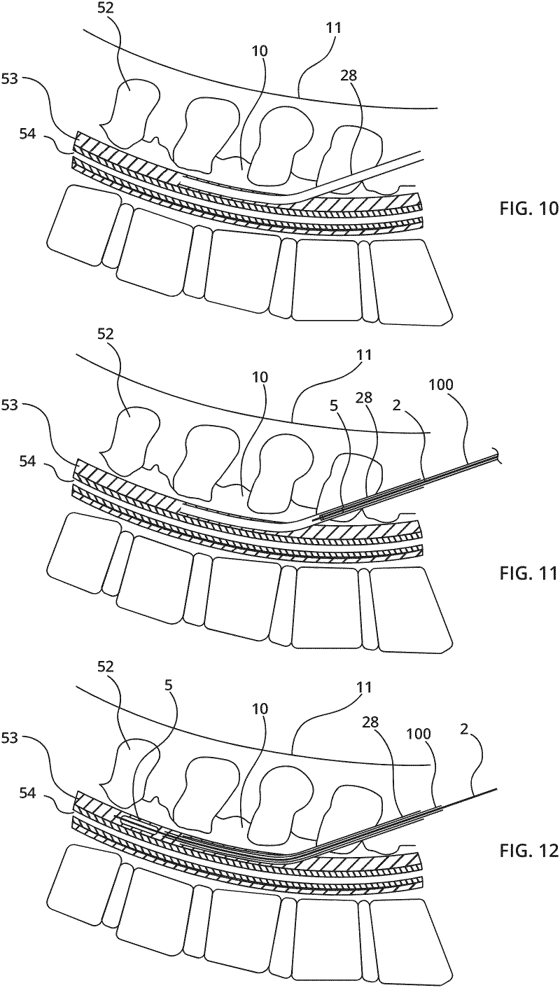

This step of the procedure aims to place the tip 64 of the outer sheath 28 somewhere along the guidewire 70 inside the patient. In some embodiments, the placement position of the outer sheath tip 64 may be at, adjacent to or directed towards the implantation site, as explained below. The distal end of the outer sheath 28 can incorporate features and materials such that its position can be determined using fluoroscopy. Once the outer sheath 28 has reached its desired position, the dilator 72 and guidewire 70 can be removed, either in succession or simultaneously, to leave the outer sheath 28 defining the pathway into the patient’s body 11, as shown in FIG. 10.

The next step in the method is to insert the delivery sheath 100, together with the device 1 comprising the lead 2 and the paddle 5, into the outer sheath 28. The paddle 5 must first be loaded into the delivery sheath 100, which may be done immediately before the insertion step or at any earlier time. The device 1 may have been delivered to the operator with the paddle 5 already received in the delivery sheath 100. However, to avoid possible damage or loss of resilience in the paddle 5, it is, in some embodiments, not stored for a long period while folded in the transport configuration.

The operator or manufacturer threads the proximal end of the lead 2 through the delivery sheath 100, passing first through the opening 102 in the distal end of the delivery sheath 100 and then through the opening 104 in the proximal end of the delivery sheath 100. The lead 2 may then be pulled until the paddle 5 is near the distal end of the delivery sheath 100, while remaining in the operative configuration. The combination of the device 1 and the delivery sheath 100 may conveniently be stored in this arrangement for a long period if required. Thereafter, as shown in FIG. 15, while holding the delivery sheath 100, the lead 2 is pulled further until the proximal end 66 of the paddle 5 engages with the distal opening 102 of the delivery sheath 100. Continued pulling on the lead 2 causes the paddle 5 progressively to fold into the transport configuration until it is fully received in the delivery sheath 100. In some embodiments, the distal tube 58 of the device 1 remains outside the delivery sheath 100.

The distal opening 102 of the delivery sheath 100 may have a simple, circular rim. Alternatively, it may comprise features (not illustrated) specifically to assist the folding of the paddle wings 21 as the paddle 5 is received in the delivery sheath 100. Such features may include the shape of the opening 102 and/or materials such as a soft, hollow core inside the opening 102 to mediate the engagement with the paddle wings 21. These features may be provided on the delivery sheath 100 itself or on a separate paddle loader (not illustrated), which is temporarily attached to the distal opening 102 while the paddle 5 is being inserted into the delivery sheath 100, then may be removed before the delivery sheath 100 is inserted into the outer sheath 28.

With the paddle 5 loaded into the delivery sheath 100, the delivery sheath 100 is then advanced into a proximal end of the outer sheath 28, as shown in FIG. 11, and slides through the outer sheath 28 until the delivery sheath 100 emanates from the distal end 64 of the outer sheath 28 inside the patient. In this way, the paddle 5 is transported to the distal end 64 of the outer sheath 28, which may be at, adjacent to or directed towards the target implantation site of the paddle 5.

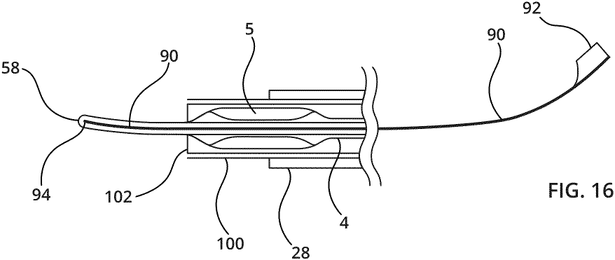

At this stage, a lead stylet 90 comprising a curved tip may be inserted into a hollow through the lead 2. It may alternatively be possible to insert the lead stylet 90 into the lead 2 before or immediately after the paddle 5 has been loaded into the delivery sheath 100. When installed, the tip of the stylet 90 engages a resilient surface within the lead 2, in some embodiments in the distal free end 58 of the lead. A proximal end of the lead stylet 90 has a finger grip 92 which the operator can use to exert an axial force on the stylet 90, whereby the tip of the lead stylet 90 will exert a force on the contact surface within the lead 2 to advance the lead 2 in the distal direction. The lead stylet 90 in some embodiments has a curved tip 94, shown in FIG. 16, which will induce a curve in the paddle distal tube 58. The orientation of the curved paddle distal tube 58 about the longitudinal axis X-X of the lead 2 can be controlled by rotating the finger grip 92 of the lead stylet 90.

With the curved paddle distal tube 58 exterior to the delivery sheath 100, the delivery sheath 100 is advanced from the distal end of the outer sheath 28 towards the desired location at or near the paddle implantation site. The steering of the delivery sheath 100 is achieved by changing the orientation of the curved paddle distal tube 58. In this scenario, the outer sheath 28 would remain stationary. It may even be possible to remove the outer sheath 28 from the patient before final positioning of the delivery sheath 100.

Once the delivery sheath 100 reaches a target location just proximal of the desired implantation site of the paddle 5, the lead stylet 90 may be pushed while the delivery sheath 100 is held stationary such that the paddle 5 is ejected from the delivery sheath 100 to occupy the desired implantation site and self-expands to its operative configuration as previously described. This is the position shown in FIG. 12. Alternatively, the delivery sheath 100 may be advanced until it reaches a target location at the desired implantation site itself. Then the delivery sheath 100 may be withdrawn while the paddle 5 is held stationary to separate the delivery sheath 100 from the paddle 5 and leave the paddle 5 occupying the desired implantation site in its operative configuration.

Once the paddle 5 is deployed, the location of the paddle 5 can be adjusted if necessary to position the paddle 5 at its intended implantation site. It can be adjusted proximally by the operator pulling on the lead 2; and it can be moved distally by the operator pushing on the paddle stylet 90. Any lateral adjustment can be made by repeatedly advancing and retracting the lead 2 while alternating the orientation of the curved distal paddle tube 58.

The orientation of the paddle 5 may be important for certain medical applications where the paddle electrodes 8 are required to be directed towards a specific location within the patient, for example towards the spinal cord 54 for SCS. Orientation control can be achieved several ways through one or a combination of the following methods.

Controlling the orientation of the paddle 5 while folded within the delivery sheath 100. Using X-ray fluoroscopy, the orientation of radio-opaque fluoro-markers 85 on the paddle 5 and/or on the lead 2 would indicate the orientation of the paddle 5 relative to the patient’s anatomy 11. Even while the paddle 5 remains stowed in the delivery sheath 100, the markers 85 can still provide information on how the paddle 5 is oriented relative to the patient. Should the marker indicate that the paddle 5 would not be deployed in the desired orientation, the operator would then twist the delivery sheath 100 within the outer sheath 28 until the desired marker orientation is achieved. Because the delivery sheath 100 is specifically designed to slide relative to the outer sheath 28, the present invention makes this operation particularly easy to perform.

Additionally or alternatively, the orientation as the paddle 5 may be controlled as it is ejected from the delivery sheath 100. While the paddle 5 is being slowly pushed out of the delivery sheath 100 (or the delivery sheath 100 is being slowly withdrawn relative to the stationary paddle 5), the paddle 5 will start unfolding, revealing fluoro-markers 85 near the distal end of the paddle 5. As the positions of the fluoro-markers 85 are known relative to another radio-opaque reference such as, but not limited to, ring electrodes on the paddle 5, the operator is able to ascertain whether the paddle 5 is starting to emerge from the delivery sheath 100 in the desired orientation. To correct the paddle 5, if needed, the operator can rotate the delivery sheath 100 by twisting the proximal end which would, in turn, rotate the paddle 5 that remains partially within it.

Finally, the orientation of the paddle 5 may still be controlled after it has exited the delivery sheath 100. In this case, the user would identify the incorrect paddle orientation after full deployment from the outer sheath. They would then pull the paddle 5 back into the delivery sheath 100 enough to achieve sufficient engagement between the paddle 5 and the delivery sheath 100. Friction between the paddle 5 and the delivery sheath 100 would then cause the paddle 5 to rotate with the delivery sheath 100 relative to the outer sheath 28. By reviewing the shape and location of the fluoro-markers 85 on the paddle 5, the operator would be able to rotate the paddle 5 until the desired fluoro-marker configuration is obtained. During this procedure, the operator may wish to repeatedly re-deploy and stow the paddle 5 to enable maximum visibility of the fluoro-markers 85.

Because it is possible to control the position of the delivery sheath 100 after it has exited from the distal end 64 of the outer sheath 28, and to control the position of the paddle 5 after it has been deployed from the delivery sheath 100, it will be understood that it is not essential (though it remains possible) for the distal end 64 of the outer sheath 28 to extend all the way to the implantation site of the paddle 5. In particular, it may be desirable that the outer sheath 28 should extend only as far into the patient as necessary to create a pathway through resistant tissue, for example by using the dilator 72 to insert the outer sheath 28. If it is possible for the delivery sheath 100, which is typically narrower and softer, to advance the remaining distance to the implantation site, then that may be, in some embodiments, preferable. For example, in the illustrated example of implanting a device 1 for SCS therapy, it may be sufficient for the outer sheath 28 only to be pushed through the ligamentum flavum until its distal end 64 has entered the epidural space 53 and is directed towards the desired implantation site, then the delivery sheath 100 carrying the paddle 5 may be advanced the remaining distance through the epidural space 53.

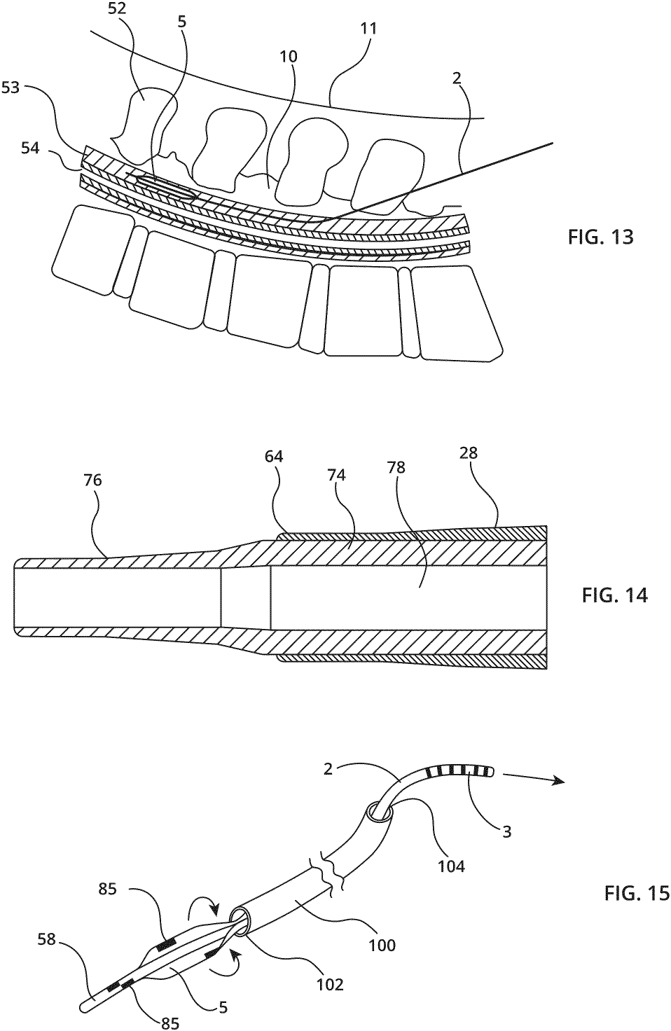

Once the paddle 5 is in place at the implantation site, the delivery sheath 100 may be withdrawn through the outer sheath 28 and out of the patient. Then the outer sheath 28 may also be withdrawn out of the patient to leave the implanted device 1, as shown in FIG. 13. In order to not compromise the position of the paddle 5 during withdrawal of the delivery sheath 100 and the outer sheath 28, the operator may hold onto the finger grip 92 of the paddle stylet 90 while pulling the delivery sheath 100 and the outer sheath 28 out of the patient. The lead 2 is in some embodiments designed to be long enough such that the operator can still hold onto the finger grip 92 after the complete withdrawal of the two sheaths 28,100 from the patient. Finally, the stylet 90 may be withdrawn from the lead 2 if desired, which will free the delivery sheath 100 and the outer sheath 28 to be removed.

When treatment has been completed or if replacement of the device 1 is required, it is necessary to remove (explant) the device 1 from the patient. In one method of explantation, the operator progressively slides the outer sheath 28 in the distal direction along the lead 2 until it reaches the desired position. The operator can then progressively slide an explantation sheath (not illustrated) through the outer sheath 28 until it reaches a position close to the paddle 5. The explantation sheath may be generally similar to the delivery sheath 100. The paddle 5 can next be pulled into the explantation sheath by pulling on the proximal end of the lead 2. This will partially or fully fold the paddle 5 into its transport configuration in the explantation sheath. Then the explantation sheath may be withdrawn through the outer sheath 28, carrying the paddle 5 inside it. Finally, the outer sheath is pulled out of the patient. In an alternative method, no explantation sheath is used and the paddle 5 is pulled directly into the outer sheath 28 for removal from the patient.

Alternatively, in some circumstances the paddle 5 may be able to be explanted without the use of a sheath by simply pulling on the proximal end of the lead 2. This technique may be particularly relevant in the case of a placement failure or after a trial implantation, as the proximal end of the lead 2 will still be emanating from the patient’s skin.

Those skilled in art may make many changes and adaptations to the embodiments described above or may replace elements with others that are functionally equivalent in order to satisfy contingent needs without however departing from the scope of the appended claims.

What is claimed is:

1. A kit comprising a device for electrotherapy and/or electrophysiology and a delivery system for the device;
wherein the device comprises:
a lead; and
a paddle comprising a paddle electrode having an exposed surface configured to come into contact with living anatomy inside a patient’s body;
the paddle being reconfigurable between a transport configuration and an operative configuration, a transverse extent of the paddle being smaller in the transport configuration than in the operative configuration; and
wherein the delivery system comprises:
a flexible, hollow outer sheath comprising a proximal end and a distal end; and
a hollow delivery sheath for receiving at least the paddle of the device in the transport configuration;
wherein the delivery sheath is configured to be inserted into the proximal end of the outer sheath and transported through the outer sheath to deliver the paddle of the device to the distal end of the outer sheath.

2. The kit according to claim 1, wherein the delivery sheath comprises a proximal end and a distal end; the distal end comprising a distal opening, through which the paddle is capable of being received and ejected.

3. The kit according to claim 2, wherein the delivery sheath further comprises a proximal opening in the proximal end of the delivery sheath, through which the lead2 of the device is capable of passing.

4. The kit according to claim 1, wherein the paddle is configured to adopt its operative configuration automatically when ejected from the delivery sheath.

5. The kit according to claim 1, wherein the device further comprises a stylet for controlling movement of the lead from outside the body.

6. The kit according to claim 1, wherein the device further comprises at least one fluoro-marker, by which the orientation of the paddle may be determined using fluoroscopy.

7. The kit according to claim 1, wherein the delivery system further comprises:
a substantially rigid, hollow needle for creating a path into the body of the patient;
a guidewire capable of passing through the needle; and
a hollow dilator capable of engagement around the guidewire and movement along the guidewire;
wherein the hollow outer sheath is capable of engagement around the dilator to move with it along the guidewire.

8. A method of delivering a device for electrotherapy and/or electrophysiology into the body of a patient;
wherein the device comprises:
a lead; and
a paddle comprising a paddle electrode having an exposed surface configured to come into contact with a living anatomy inside a patient’s body;
the paddle being reconfigurable between a transport configuration and an operative configuration, a transverse extent of the paddle being smaller in the transport configuration than in the operative configuration; and
wherein the method comprises:
inserting a flexible, hollow outer sheath into the body of the patient such that a distal end of the outer sheath is at or directed towards an implantation site inside the patient’s body;
inserting at least the paddle of the device into a hollow delivery sheath with the paddle in the transport configuration;
inserting the delivery sheath into a proximal end of the outer sheath and transporting the delivery sheath through the outer sheath to deliver the paddle of the device to the distal end of the outer sheath; and
ejecting the paddle from the delivery sheath.

9. The method according to claim 8, wherein the delivery sheath comprises a proximal end and a distal end; and wherein the insertion of the paddle into the delivery sheath and the ejection of the paddle from the delivery sheath are carried out through a distal opening in the distal end of the delivery sheath.

10. The method according to claim 9, further comprising the step of passing the lead of the device through a proximal opening in the proximal end of the delivery sheath.

11. The method according to claim 8, wherein the paddle adopts its operative configuration automatically when ejected from the delivery sheath.

12. The method according to claim 8, comprising the preliminary steps of:
using a hollow needle to create a path into the body of the patient;
inserting a guidewire through the needle and directing the guidewire such that a distal end of the guidewire is at or directed towards the implantation site; and
withdrawing the needle;
and wherein the step of inserting the outer sheath comprises:
engaging the outer sheath around a hollow dilator;
engaging the dilator around the guidewire;
moving the dilator and the outer sheath along the guidewire until a distal end of the dilator is at or directed towards the implantation site; and
withdrawing the dilator through the outer sheath.

13. The method according to claim 8, wherein the step of ejecting the paddle from the delivery sheath comprises determining the position or orientation of the paddle by using fluoroscopy to observe at least one fluoro-marker on the device.

14. The method according to claim 8, wherein the step of ejecting the paddle from the delivery sheath comprises, while the paddle remains at least partially within the delivery sheath, rotating the delivery sheath to adjust the orientation of the paddle.
